(19) United States
(12) Patent Application Publication
(10) Pub. No.: US 2011/0081396 A1
(43) Pub. Date: Apr. 7, 2011

(54) GLASS CERAMIC SCAFFOLDS WITH COMPLEX TOPOGRAPHY

(75) Inventor: Isabelle L. Denry, Dublin, OH (US)

(73) Assignee: THE OHIO STATE UNIVERSITY RESEARCH FOUNDATION, Columbus, OH (US)

(21) Appl. No.: 12/878,282

(22) Filed: Sep. 9, 2010

Related U.S. Application Data
(60) Provisional application No. 61/240,803, filed on Sep. 9, 2009.

(51) Int. Cl. A61F 2/00; A61P 19/00; C04B 35/64

(52) U.S. Cl. 424/423; 264/666

(57) ABSTRACT

A bioactive and bioresorbable scaffold including a glass-ceramic material including fluoroapatite and hydroxyapatite doped with about 1-5 wt.% niobium oxide that is shaped into a scaffold is described. The glass-ceramic material has high crystallinity and a complex topography which provide it with greater structural strength and bioresorbability. Methods of preparing the bioactive and bioresorbable scaffold and methods of using the scaffold for musculoskeletal engineering are also provided.

### CROSS-REFERENCE TO RELATED APPLICATIONS

This application claims priority to U.S. Provisional Patent Application Ser. No. 61/240,803, filed Sep. 9, 2009, which is incorporated herein by reference

### GOVERNMENT FUNDING

The present invention was made with government support by the NIH-NIDCR under Grant No. R01 DE019932-01. The Government may have certain rights in this invention.

### BACKGROUND

Numerous biomaterials are available for bone grafts in oral and maxillo-facial surgery. They include autografts, allografts, xenografts and a wide variety of synthetic materials. Autografts are often referred to as the “gold standard”; bone is usually harvested from a donor site such as the iliac crest. Autogenous bone possesses all the characteristics necessary for producing new bone; it is osteogenic, osteoconductive and osteoinductive. Success rates are high but one obvious drawback of the autograft is the associated morbidity, including the possibility of recurrent pain, risk of infection, cost of a second surgery and the fact that autogenous bone is not always available in sufficient quantity or acceptable quality. This is true, for example, with patients that suffer from osteoporosis.

An alternate solution is to use an allograft, available from bone banks. In addition to the reluctance of many patients to having bone harvested from human cadavers grafted in their own body, the associated risks are still unclear. Despite the stringent preparation guidelines and rigorous donor screenings, the risk of human immunodeficiency virus (HIV) transmission alone with allograft bone is 1 case in 1.6 million population. Boyce et al., Orthop Clin North Am, 30, 571-581 (1999). A case of hepatitis B transmission and three cases of hepatitis C transmission have been reported with allograft tissue. Tomford W W, J Bone Joint Surg Am, 77, 1742-1754 (1995); Conrad et al., J Bone Joint Surg Am., 77, 214-224 (1995). Reports from the Center for Disease Control and Prevention revealed more recently that other diseases have been transmitted via allografts. Transmission of the much-feared Creutzfeldt-Jakob disease cannot be entirely excluded. Several allografts products were recalled in 2005 by the Food and Drug Administration. Another problem with the use of allografts is that the infection control and sterilization procedures greatly reduce the osteoinductivity of the bone tissue.

The last option available for bone-like graft materials is a xenograft such as bovine-derived anorganic bone. A recognized disadvantage of xenografts is that they often exhibit unpredictable resorption rates. Hile et al., Biomaterials in orthopedics. Yaszemski M, ed., New York: Marcel Dekker, Inc., p 185-194 (2004). In addition, clinical studies with xenografts have yet to demonstrate better tissue response and bone formation, compared to autografts or allografts.

One way to avoid some of the drawbacks of the bone graft materials described above is to use synthetic biomaterials such as ceramic composites or ceramic/polymer composites. Ceramic composites include calcium sulfate cements, calcium phosphate-based sintered ceramics and cements, and bioactive glasses and glass-ceramics. Calcium phosphates have been studied extensively and used as synthetic bone graft materials. LeGeros et al., “Bioceramics: Calcium phosphate ceramics: past, present and future”, Trans Tech Publications, Ben-Nissan B, ed.; Sydney, p 3-10 (2002). The most popular calcium phosphates used as bone graft materials are beta tricalcium phosphate (β-TCP) and hydroxyapatite (HAp), which can be either entirely synthetic or of coralline origin β-TCP has been shown to have a higher resorption rate than HAp, which could lead to failure of the bone graft if this rate exceeds the rate at which new bone can be fowled. Koerten et al., J Biomed Mater Res., 44, 78-86 (1999) Both β-TCP and HAp are mostly used in particle form or as coatings due to the difficulty of sintering in bulk form, together with the thermal instability of both ceramics. Another drawback of calcium phosphate ceramics lies in their mediocre mechanical properties, compared to both cancellous and cortical bone. Rezwan et al., Biomaterials, 27, 3413-3431 (2006).

The concept of bioactivity was discovered and developed almost four decades ago. A material is considered biologically active when “an interfacial bond forms between the tissues and the implant”. Bioactive glasses can be used clinically in bulk, particle form, and more recently, as scaffolds. Hench L L., J Am Ceram Soc., 81, 1705-28 (1998). Their exclusive advantage is the rapid reaction rate between the glass surface and the surrounding tissues. Ionic diffusion at the surface of the bioactive glass leads to heterogeneous nucleation and growth of hydroxycarbonate apatite (HCA). This later promotes cell attachment and differentiation, and ultimately bone formation. It is now well established that there is broad range of compositions for which glasses and glass-ceramics are bioactive. Hench et al., “Bioactive glasses”, An introduction to bioceramics, Hench L L, Wilson J, eds. River Edge, N.J.: World Scientific, p 41-62 (1993).

Fluoroapatite has been considered for use as a bioactive glass-ceramic. Fluorapatite (FAp) is chemically and structurally similar to hydroxyapatite (Hap), the substitution of fluorine for hydroxyl is associated with a contraction along the a-axis of the unit cell while the c-axis remains unchanged. FAp is less soluble than HAp, but it is also more stable chemically and easier to synthesize as a stoichiometric compound. Additionally, FAp can provide fluoride release at a controlled rate and several studies have demonstrated the stimulating effect of fluoride on bone formation. Lauet et al., J Bone Miner Res., 13, 1660-1667 (1998) A bioactive glass-ceramics containing both FAp and mica has been commercialized under the name Bioverit® and was first developed by Holand. Holand W., J Non Cryst Solids, 219, 192-197 (1997). Fluorapatite and mica crystallization occur in a “two-fold controlled mechanism.” Homogeneous nucleation of FAp occurs in the temperature range of 750-1000° C. The microstructure can be varied from droplet-shaped FAp crystals in the 300-700 nm range to a final microstructure comprising both mica and FAp, depending on the heat treatment applied. Moisescu et al., J Non Cryst Solids, 248, 169-175 (1999).

Bone graft materials are typically porous. The scaffolds are macroporous and exhibit either an interconnected pore structure or a closed pore structure, depending on the fabrication technique. Interconnected pore structures provide a number of advantages. However, an interconnected pore structure is not achieved easily with the porogen approach. Other approaches involve more modern rapid prototyping techniques such as stereolithography, laser sintering, 3-D printing or fused deposition modeling. Stevens et al., Journal of Biomedical Materials Research Part B: Applied Biomaterials, 85, 573-582 (2008). These approaches, although successful in producing porous scaffolds are sometimes costly and do not always lead to the interconnected porosity that is necessary for successful osteoconduction. Moreover, in the case of salts as pore-formers, the control of the pore-former elimination can be difficult and remaining impurities are detrimental to the bioactivity of the scaffold. Sol-gel derived bioactive glass scaffolds produced by addition of various porogens have shown high potential for use in bone tissue engineering applications. Sepulveda et al., J Biomed Mater Res, 59, 340-348 (2002). One drawback of a sol-gel approach is that processing is fairly complex and control of the pore size and interconnectivity technically delicate.

As mentioned earlier, a unique advantage of bioactive glass-ceramics as scaffold materials is that the final microstructure and therefore the mechanical properties can be controlled by crystallization heat treatment. It has been reported that some bioactive glass compositions undergo crystallization prior to significant sintering and densification of the porous scaffold. Clupper et al., J Non-Cryst Solids 318, 43-48 (2003). However, Chen et al. have recently demonstrated that bioactive glass scaffolds can be successfully fabricated by optimizing the sintering schedule so as to obtain dense scaffolds, together with the formation of fine crystals as a reinforcing phase. Chen et al., Biomaterials, 27, 2414-2425 (2006). Moreover, it was shown that bioactivity was preserved while the resorbability of the scaffold could be tailored by controlling the amount of crystallization. Chen et al., Journal of Biomedical Materials Research Part A 84A, 1049-1060 (2008). Although bioactive glass-ceramics are attractive as synthetic scaffold materials, their clinical applications are limited by their low compressive strength and lack of mechanical integrity. In addition, existing glass-ceramic materials typically have a relatively smooth surface with a low surface area which is fundamentally different from natural bone, which has a high surface area. There is a need for a bioactive and bioresorbable synthetic scaffold that exhibits a compressive strength similar or greater to that of cancellous bone and that also provides good resorbability and bioactivity.

### SUMMARY OF THE INVENTION

The present invention provides a bioactive and bioresorbable glass ceramic scaffold with improved properties over glass ceramic scaffolds in the prior art. The glass-ceramic material used to form the scaffold has a complex topography that enables the material to more closely resemble bone and to increase the bioresorbability of the scaffold. The glass-ceramic material also has high crystallinity which provides a scaffold with greater strength.

Accordingly, one aspect of the invention provides a bioactive and bioresorbable scaffold formed from a glass-ceramic material shaped into a scaffold, in which the glass-ceramic material includes fluoroapatite and hydroxyapatite doped with about 1-5 wt. % niobium oxide, and wherein the glass-ceramic material has high crystallinity and a complex topography. In some embodiments, the glass-ceramic material includes an outer layer that includes strontium. In additional embodiments, the glass-ceramic material includes an interconnected porous network.

An additional aspect of the invention provides a method of musculoskeletal engineering that includes positioning a bioactive and bioresorbable scaffold of the invention in a subject to provide structural support for nearby tissue. In some embodiments, the method is used in oral or maxillo-facial surgery.

A further aspect of the invention provides a method of making a bioactive and bioresorbable scaffold that includes the steps of melting suitable reagent grade oxides and carbonates together with niobium oxide at a temperature from about 1450 to 1600° C. to obtain a glass-ceramic material including 28-38% SiO₂, 12-18% CaO, 12-18% MgO, 11-17% Al₂O₃, 1-3% Na₂O, 5-8% K₂O, 4-6% F, 10-14% P₂O₅, and 1-5% Nb₂O₅, and then allowing the glass-ceramic material to cool. The glass-ceramic material is then ground to a powder and the glass-ceramic material is remelted at a temperature from about 1450 to 1600 C to homogenize the glass ceramic material, after which it is again allowed to cool. The glass-ceramic material is then ground again to a powder and the powder is compacted and sintered at a temperature from about 750 to about 1100° C. and allowed it to cool to foim a glass-ceramic scaffold. In further embodiments of the method, the glass-ceramic material is sintered over a polymeric foam suitable for forming a porous glass-ceramic scaffold. The polymeric foam can include a pre-coat to improve the strength of the resulting porous glass-ceramic material. In an additional embodiment, the method also includes providing the scaffold with an outer layer including strontium by ion-exchange.

In a further aspect, the present invention provides a bioactive and bioresorbable scaffold prepared according to any one of the methods of the invention.

### BRIEF DESCRIPTION OF THE FIGURES

FIG. 1 provides a scanning electron microscope image of finely dispersed nanosized FAp crystals surrounding larger crystals with a small amount of forsterite in a glass-ceramic doped with 1 wt. % Nb₂O₅ (950° C./2 hrs).

FIG. 2 provides a characteristic compression graph for an embodiment of the glass-ceramic scaffold of the present invention.

FIG. 3 provides graphs showing the percentage of weight loss as a function of time for strontium-substituted apatite glass ceramics, with 3A showing the weight loss for disc shaped specimens, and 3B showing the weight loss for a scaffold specimen.

### DETAILED DESCRIPTION OF THE INVENTION

The present invention provides a bioactive and bioresorbable scaffold in which the glass-ceramic material that is used to form the scaffold has high crystallinity and a complex topography. The high crystallinity provides improved structural characteristics such as strength, while the complex topography allows the material to more closely resemble bone and encourages bioresoprtion of the scaffold.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

Also herein, the recitations of numerical ranges by endpoints include all numbers subsumed within that range (e.g., 1 to 5 includes 1, 1.5, 2, 2.75, 3, 3.80, 4, 5, etc.).

In one aspect, the present invention provides a bioactive and bioresorbable scaffold. The scaffold formed from a glass-ceramic fluorapatite-based material shaped to form a scaffold. An example of a fluoroapatite-based material is a mixture of fluoroapatite and hydroxyapatite. A fluorapatite-based material is a calcium-phosphate-based ceramic material in which a significant portion of the material is fluorapatite. For example, the fluorapatite-based material can be a calcium-phosphate-based ceramic in which 50%, 60%, 70%, 80%, or 90% of the calcium-based ceramic material is fluoroapatite.

The fluorapatite-based material forming the scaffold can be doped with niobioum oxide. For example, a glass-ceramic material made of fluoroapatite and hydroxyapatite can be doped with from about 0.5 to 10 wt % niobium oxide, or more preferably about 1-5 wt. % niobium oxide. Glass-ceramics in the system CaO—Nb₂O₅—P₂O₅ have been synthesized and tested in vitro, and these studies showed that the niobium-containing calcium phosphate ceramics exhibited a good biocompatibility. Gross et al., Bioceramics 14; p 165-168 (2002). Bulk crystallization of nanocrystals can occur with or without phase separation in niobium-containing glass ceramics, depending on the composition, and crystallization takes place in high-niobiate phase regions. Petrovskii et al., Glass Phys Chem., 29, 243-253 (2003). The atomic radius and electronegativity of the niobium cation are very similar to those of zirconium and titanium. The present inventors have shown that additions of Nb₂O₅ to fluorapatite glass-ceramic compositions, led to a microstructure consisting of nanosized spherical FAp crystals in the 150 to 300 nm range, while the niobium-free base composition exhibited needle-shaped FAp crystals, 2 μm in length. Denry et al., Journal of Biomedical Materials Research Part B-Applied Biomaterials, 75B, 18-24 (2005) While not intending to be bound by theory, Nb₂O₅ appears to induce phase separation in the composition range tested, and secondary crystallization occurs, leading to nanosized FAp crystals within phase separated droplets, which aids in providing a complex topography.

The niobium-doped fluorapatite-containing glass-ceramic material of the present invention also has high crystallinity. The high crystallinity is provided by the chemical composition and the heat treatment used to prepare the glass-ceramic material. High crystallinity indicates that the glass-ceramic material is at least 50% crystalline, with additional embodiments having at least 55%, at least 60%, or at least 65% crystallinity. The level of crystallinity contributes to the mechanical properties of the glass-ceramic material, with higher levels of crystallinity corresponding to higher strength. For example, embodiments of the the glass-ceramic material have a flexural strength of at least about 100 MPa, a modulus of elasticity of at least about 80 GPa, and a fracture toughness of at least about 1.2 MPa·m^{0.5}.

The niobium-doped fluorapatite-containing glass-ceramic material of the present invention also provides a complex topography. Implant substrate topography plays an important role in cell and tissue structure and function, and micro and nano-topography can be used to control and enhance cell reactions to a material surface. Wood, M., Journal of the Royal Society Interface, 4, 1-17 (2007). Accordingly, the complex topography of the glass-ceramic material of the invention helps provide a surface that is bioactive. A material is considered to be bioactive when it is capable of forming an interfacial bond between the tissues and the implant. In other words, the bioactive surface leads to integration of the implant in the surrounding tissue, rather than merely being neutral or non-reactive. A bioactive surface therefore can be expected to bond to one or more extracellular matrix proteins present on the cells in the surrounding tissue. This bonding integrates the material, and can also stimulate the activity of nearby cells. For example, embodiments of the invention provide a surface that that stimulates osteogenesis. This may be stimulated through binding to osteoblasts. Osteogenesis has the beneficial effect of building new natural tissue to replace the scaffold as it is resorbed.

The inorganic component of natural bone consists mainly of partially carbonated HAp nanocrystallites. The size of these crystallites determines the mechanical properties of natural bone. Studies have shown that bone grafts with nano-topographic surfaces have better implant-tissue integration. Moreover, enhanced long-term osteoblast functions have been reported, when cultured on nano-phase ceramics. Webster et al., Biomaterials, 21, 1803-1810 (2000). Recent work on nanosized hydroxyapatite (NHAp) ceramics prepared by spark plasma sintering showed that osteoblast density after only 90 minutes of incubation was much higher on NHAp than on microstructured HAp (MHAp). Matrix mineralization at 7 and 14 days was also much higher on NHAp than on MHAp. Guo et al., Journal of Biomedical Materials Research Part A, 82A, 1022-0132 (2007). It has also been shown that the bone resorption surface created by osteoclasts exhibits three-dimensional complexity at the sub-micron scale range. Davies, J., J. Dent. Educ., 67, 932-949 (2005). Surface micro-topography is also responsible for platelet activation regardless of the presence of Ca and PO₄ at the implant surface. Kikuchi et al., Biomaterials, 26, 5285-5295 (2005). Furthermore, Mendes et al. showed that a traditionally non bone-bonding metallic material can be rendered bone-bonding by the formation of a three-dimensionally complex surface by deposition of nanosized calcium phosphate crystals. Mendes et al., Biomaterials, 28, 4748-4755 (2007).

The term “complex topography,” as used herein, refers to a surface that includes nanoparticles having substantially different sizes and/or morphology. Topography refers to surface features, in this context, while the complex nature of the topography refers to the fact that the surface is non-uniform. A complex topography is advantageous in part because it more closely resembles actual bone mineral crystal, which has a large surface area, thereby facilitating bone remodeling as the glass-ceramic material is bioresorbed. The complex topography also provides particles with a significant density. Particles provided with an insufficient density would not be present at high enough levels to prevent the surface from behaving as a substantially flat and uniform surface. For example, embodiments of the invention provide scaffold surfaces having a surface density of from about 1 to about 50 crystals per square micrometer. Further embodiments provide a surface density from about 2 to about 25 crystals per square micrometer, or from about 10 to about 25 crystals per square micrometer. The crystals present on the scaffold surface include nanosized fluoroapatite crystals.

While the complex topography of the glass-ceramic material is visible at the surface of the glass-ceramic material, it should be noted that this architecture is present throughout the material. As a result, as the glass-ceramic material is resorbed and new surfaces are formed, these surfaces will also exhibit the complex topography. This has been demonstrated by etching the material to reveal the complex topography, grinding the material to remove a significant amount, and then re-etching, which again reveals a surface with complex topography.

The crystals forming the complex topography are nanosized crystals. In some embodiments, the nanosized crystals have a size ranging from about 50 to about 400 nanometers, or from 50 to about 200 nanometers. In additional embodiments, the crystals fall into specific differing size groups. For example, in another embodiment, the complex topography comprises one group of smaller crystals having a size less than about 300 nanometers, and another group of larger crystals having a size greater than about 300 nanometers. Embodiments of small crystals include those having a size ranging from about 50 to about 300 nanometers, from 60 to about 200 nanometers, from about 70 to about 150 nanometers, or from with from about 80 to 100 nanometers. The larger crystals can have a size ranging from about 300 to about 1000 nanometers, from about 300 to about 700 nanometers, or from about 300 to about 500 nanometers. The size differences can be caused by the mineral nature of the crystals. For example, the small crystals having a size from about 80 to 100 nanometers can be fluorapatite crystals, whereas the larger crystals can be forsterite crystals.

The glass-ceramic material of the invention can also include an outer layer including strontium. Strontium (Sr) has generated considerable interest as a substitute ion for calcium in hydroxyapatite crystals due to its reported efficacy for preventing bone resorption in the treatment of osteoporosis. Marie P., Current Opinion in Pharmacology 5, 633-636 (2005) Strontium is primarily incorporated by ion-exchange onto the apatite crystal surface in newly formed bone. However, when administered orally, even at high doses (3 mmol. Sr per day for 13 weeks), less than one calcium ion can be substituted by Sr in the apatite structure. As a result, several research groups have focused on the development of Sr-substituted apatite-based ceramics for biomedical applications. Landi et al., Acta Biomaterialia, 3, 961-969 (2007) Strontium-substituted apatite can be synthesized by various methods including precipitation, hydrolysis and solution-mediated reactions. Bigi et al., Inorganica Chimica Acta, 360, 1009-1016 (2007) The progressive substitution of strontium for calcium in the HAp structure is associated with a linear increase in the lattice constants, due to the slightly larger ionic radius of strontium. Collin R., J Am Chem Soc., 81, 5275-5278 (1959) The substitution is also associated with the creation of lattice defects, vacancies and distortions which affect the surface properties in terms of hydration layers and surface charges. Landi et al., Acta Biomaterialia, 3, 961-969 (2007)

Since strontium acts as a network modifier in silicate glasses, it was thought that adding strontium to the bulk glass composition might impair the crystallization kinetics of fluorapatite glass-ceramics, preventing the formation of nanosized crystals. Conuier et al., Physical Review B, 59, 13517-13520 (1999). Ion-exchange is a technique that can be used to strengthen glasses and glass-ceramics. Nordberg et al., J Am Ceram Soc, 47, 215-219 (1990). The process is diffusion-driven and involves the exchange of ionic species between a molten salt bath and a bulk glass or glass-ceramic. Strengthening is obtained by the replacement of ionic species with other species with larger ionic radius, thereby creating compressive stresses at the glass or glass-ceramic surface as the ion-exchange is conducted below the glass transition temperature. The inventors have determined that full substitution of strontium for calcium in microcrystalline HAp can be achieved by ion-exchange at 900° C., by using strontium nitrate as the exchanging salt. A partial substitution of calcium for strontium in the niobium-doped bioactive glass-ceramic can also be achieved at lower temperatures (700 and 750° C.). Several experimental parameters can be adjusted to achieve the desired level of strontium replacement. These include the choice of molten salt and the reaction temperature and time.

Providing an outer layer in which at least a portion of the calcium has been replaced with strontium provides one or more advantages for the glass-ceramic material. Depending on the desired level of strontium replacement, the outer layer including strontium can vary in thickness. For example, the outer layer including strontium can have a thickness from about 1 to about 50 micrometers. Typically the outer layer including strontium has a thickness from about 5 micrometers to about 25 micrometers. One advantage is that a scaffold built of glass-ceramic material with an outer layer of strontium will gradually elute strontium into the local environment, which can have a beneficial effect on the surrounding tissue. Another advantage is that an outer layer of strontium can increase the solubility of the glass-ceramic material, thereby increasing the bioresorption of the material. The extent of ion-exchange of strontium for calcium in the crystalline component of the glass-ceramic material can be controlled by adjusting the ion-exchange parameters. This in turn regulates the resorption rate of the glass-ceramic material and/or the rate of release of strontium into the surrounding environment when the material is positioned in vivo.

A preferred use of the glass-ceramic material of the invention is use as a scaffold. A scaffold, as the term is used herein, is a constructed material that is intended for use as a tissue replacement, and in particular a temporary tissue replacement Due to their similarity to natural bone tissue, glass-ceramic scaffolds of the present invention are particularly suitable for the replacement of bone and bone-like materials such as cartilage. The glass-ceramic material can be molded or otherwise shaped during preparation to have any desired configuration. Typically, the glass-ceramic material is molded to have the shape of the bone or bone-like material that it is being substituted for. Alternately, the glass-ceramic material can be configured into an artificial shape that provides the support needed for a particular type of surfer. For example, the scaffold material of the present invention can be configured for restoring or regenerating bone, cartilage, muscle, or musculoskeletal tissue. The scaffold material can also be used for cosmetic work or “bioengineering,” where a support structure is provided for the creation of new tissue rather than the replacement or regeneration of existing tissue.

The scaffolds of the present invention are bioresorbable. Bioresorbable, as used herein, refers to the ability of the scaffolds to be gradually degraded by physiological processes in vivo, to allow the replacement of the glass-ceramic material with native tissue. For example, if the scaffold is used to replace bone, the scaffold may be gradually degraded while osteoblasts rebuild bone tissue in its place (i.e., bone remodeling). Factors involved in bioresorption typically include physiological dissolution, which depends on pH and the nature of the glass-ceramic composition, physical/mechanical disintegration, and biological degradation by means such as phagocytosis. The complex topography of the glass-ceramic materials of the present invention facilitate their bioresorption in part due to their similarity to natural bone tissue, which also has a complex topography which facilitates binding and resorption by osteoclasts. Preferably, the rate of biological degradation of the scaffold material occurs at a rate similar to the growth rate of new tissue, to avoid production of a gap at the interface between the scaffold material and the newly grown bone tissue.

The glass ceramic material of the invention may be porous. A porous material includes numerous gaps or “pores” in the material. The advantages of using porous scaffolds are numerous; for example, a porous graft will resorb significantly faster than solid grafts of equivalent volume due to the high surface area. In addition, a porous graft material will allow for more rapid vascularization and ingrowth of new bone. The pore size and its control are important factors. A recently published computational multiscale approach demonstrated that bone regeneration increased as a function of pore size. Sanz-Herrera et al., Acta Biomaterialia, 5, 219-229 (2009) Accordingly, embodiments of the glass-ceramic material include pore sizes ranging about 1 micrometer to about 1 millimeter, or from about 100 to 500 micrometers. In addition, the pores can make up a varying percentage of the glass-ceramic material. For example, the pores can make up anywhere from 10% to 90% of the volume of the glass ceramic, with a total porosity of about 75 to 85% being preferred. Use of a porous material also provides the advantage of decreasing the weight of a scaffold prepared from the glass-ceramic material, while retaining a relatively high level of mechanical strength.

In additional embodiments of the invention, the glass-ceramic material includes an interconnected pore structure. Interconnected pore structures include pores that are connected to one another to form channels that make up a three-dimensional interconnected structure within the material, rather than being discrete and separate spaces within the material. The advantage of an interconnected pore structure is that the scaffold is more permeable to body fluids and more easily colonized by cells throughout. Since the interconnected pore structure is more osteoconductive, it can be more readily bioresorbed and replaced with natural bone.

Further aspects of the invention provide a method for tissue or musculoskeletal tissue engineering that include in vivo placement of a bioactive and bioresorbable scaffold as described herein for bioengineering, restoring or regenerating bone and/or other tissue, wherein the bone and/or other tissue is, at least in part, bioengineered, restored or regenerated. In particular aspects of the method, bioengineering, restoring or regenerating bone or another tissue is in vitro or ex vivo, including placement under body fluid conditions. The method includes positioning a bioactive and bioresorbable scaffold in a subject to provide structural support for nearby tissue. In particular embodiments of the method, the compositions are used for dental and orthopedic implants, craniomaxillofacial applications and spinal grafting, and said composition is suitable to promote bone in-growth and repair. In particular, the scaffolds can be used in oral or maxillo-facial surgery.

A further aspect of the invention provides a method of making a bioactive and bioresorbable glass-ceramic material, and in particular a bioactive and bioresorbable scaffold. This method includes a number of steps. First, suitable reagent grade oxides and carbonates are melted together with niobium oxide at a temperature from about 1450 to 1600° C. to obtain a glass-ceramic material. The composition can be heated in covered platinum crucibles to decrease fluorine losses, and an excess of fluoride in the initial composition can also be used to offset fluorine loss by volatilization. The composition of the glass material can vary to some extent depending on the starting materials, and will include compositions within the ranges of 28-38% SiO₂, 12-18% CaO, 12-18% MgO, 11-17% Al₂O₃, 1-3% Na₂O, 5-8% K₂O, 4-6% F, 10-14% P₂O₅, and 1-5% Nb₂O₅. The glass-ceramic material is heated at this temperature for 1 to 5 hours, with heating for about 3 hours being preferred.

After heating, the glass-ceramic material is allowed to cool. The glass-ceramic material is then ground to a powder, or otherwise physically disrupted to provide small fragments. For example, the powdering can be accomplished using a planetary mill. The fragments (e.g., powder) of the glass-ceramic composition are then remelted, again at a temperature from about 1450 to 1600 C in order to better homogenize the glass ceramic material. The glass-ceramic material is again heated at this temperature for 1 to 5 hours (e.g., 3 hours) and then allowed to cool. The glass-ceramic material is then treated to form a powder by a technique such as grinding. The powder is then compacted and sintered at a temperature from about 750 to about 1100° C. and allowing it to cool to form a glass-ceramic scaffold. The configuration of the scaffold can be created in a number of ways. For example, it can be determined based on the shape into which the powder is compacted before heating. Alternately, the shape can be varied by mechanical processing such as grinding after the glass-ceramic material has been sintered.

The glass-ceramic scaffold is preferably made using a glass-ceramic material that is porous. The porosity can be introduced into the scaffold using a variety of techniques known to those skilled in the art. For example, porosity can be introduced by foaming of ceramic suspensions, or swelling of ceramic bodies via gas evaporating chemical reactions from organic or inorganic sources, the porogens being later eliminated. Sopyan et al., Science and Technology of Advanced Materials, 8, 116-123 (2007) Porogens include salts and microspheres of various polymers and biopolymers. These techniques typically provide a material with a closed porous structure.

In other embodiments of the invention, the glass-ceramic material is provided with an interconnected porous structure. A number of techniques for providing an interconnected porous structure are known. An preferred approach to produce bioactive ceramic scaffolds for the present invention is to use a polymeric sponge technique to obtain a macroporous interconnected scaffold after elimination of the polymeric template and sintering. Pu et al., Journal of the American Ceramic Society, 87, 1392-1394 (2004). The main advantages of this technique are its simplicity, reliability, the ability to carefully control the chemistry of the final product by complete elimination of all impurities, and most importantly, a controllable pore size and the possibility of fabricating a variety of shapes. Unfortunately, the ceramic foams produced by the polymer sponge technique are not as strong as would be preferred. This appears to be due to the fact that, after elimination of the polymeric template, the triangular cross-section of the struts is hollow, with sharp apices. In addition, the struts often present longitudinal cracks.

Problems associated with the standard form of the polymeric foam technique can be resolved by depositing a pre-coating on the polymeric template, thereby eliminating sharp apices in cross section. This technique was successfully demonstrated by Jun et al., who used a fugitive carbon slurry to pre-coat polyurethane struts. Jun et al., Journal of the American Ceramic Society, 89, 2317-2319 (2006) The compressive strength materials prepared using pre-coated specimens was about twice that of the non pre-coated control specimens. Pu et al. used a silica sol to pre-coat a polyurethane foam and obtained a more uniform and thicker slurry coating. Pu et al., J Am Ceram Soc.; 90, 2998-3000 (2007). Surface active agents and carboxymethyl cellulose have also been used to provide a good polymeric coating for a foam Liu et al., Journal of Inorganic Materials, 21, 1185-1190 (2006). These techniques can be used to provide glass-ceramic materials including an interconnected porous structure with higher mechanical strength.

In aspects of the invention using a polymeric foam, the method includes the following additional steps. A foam with the desired level of pores per inch (e.g., 50 pores per inch) is prepared. A suitable material for the foam is polyethylene. In some embodiments, the glass ceramic material is then sintered over the polymeric foam. Alternately, the foam can be provided with pre-coating to smooth the sharp apices within the foam before sintering. For example, the foam can be pre-coated with carboxymethyl cellulose or silica sol. In this embodiment, the powdered glass ceramic material is prepared as a slurry by dissolving it in solution, and then loading it onto the pre-coated polymeric foam, which is then dried and sintered to prepare a porous glass-ceramic material.

In another aspect, the method of preparing the glass-ceramic scaffold includes the step of provided the glass-ceramic scaffold with an outer layer including strontium by ion-exchange. For example, the ion-exchange can be carried out using molten strontium nitrate at a temperature from about 650 to about 800° C. Alternately, the ion-exchange is carried out under somewhat milder conditions using a mixture of molten strontium nitrate and strontium dinitrate at a temperature from about 550 to about 650° C. By varying the time and temperature during which ion-exchange is carried out, the solubility of the glass-ceramic scaffold can be varied, and the ability of the scaffold to deliver strontium to the local environment can be changed. For example, the release of strontium and the solubility of the scaffold can be increased by increasing the depth of the outer layer including strontium.

Aspects of the invention provide a bioactive and bioresorbable scaffold prepared according to any of the methods described herein. Such a bioactive and bioresorbable scaffold will include a glass-ceramic material including fluoroapatite and hydroxyapatite doped with about 1-5 wt. % niobium oxide that has high crystallinity and a complex topography. Use of a proper method of preparation can be very important for obtaining a glass-ceramic scaffold having the desired traits of high crystallinity and complex topography.

The present invention is illustrated by the following examples. It is to be understood that the particular examples, materials, amounts, and procedures are to be interpreted broadly in accordance with the scope and spirit of the invention as set forth herein

### EXAMPLES

### Example 1

### Preparation of Bioactive Fluorapatite (FAp) Glass-Ceramics Containing Nanocrystals

The effect of addition of niobium oxide from 0 to 5 wt. % on the microstructure of glass-ceramics derived from the Bioverit® base composition was evaluated. Fluorapatite-based glasses doped with either 1, 2.5 or 5 wt. % niobium oxide and with decreasing amounts of magnesium oxide were prepared. The niobium-free parent glass composition is given in Table 1. Reagent grade alkali carbonates were used to ensure adequate homogenization of the glasses during melting. The batch ingredients were tumbled for 4 h in a shaker-mixer, melted at 1525° C. for 3 h in platinum crucibles and quenched in water. Covered platinum crucibles are used to limit fluorine losses by volatilization. After quenching, the frits were powdered in a planetary mill and re-melted at 1525° C. for 3 h to ensure homogeneity. The molten glasses was cast into stainless-steel molds to form 12×60-mm cylindrical ingots, transferred to an oven set at 600° C. and furnace-cooled to room temperature. Bulk specimens of each glass (n=3 per group) were polished and carbon-coated prior to SEM and EDS analysis to assess the chemical composition of the glasses

TABLE 1
Parent Glass Composition

| Component | Weight % |
|---|---|
| SiO₂ | 30.5 |
| CaO | 14.4 |
| MgO | 14.8 |
| Al₂O₃ | 15.9 |
| Na₂O | 2.3 |
| K₂O | 5.8 |
| F | 4.9 |
| P₂O₅ | 11.4 |

The inventors demonstrated that adding small amounts of Nb₂O₅ led to the crystallization of FAp nanocrystals (200-300 nm in diameter). Nb₂O₅ appeared to promote phase separation and crystallization of nanosized crystals. The percent crystallinity was 36%, as determined by quantitative stereology on digital micrographs. The biaxial flexural strength of the glass-ceramic doped with 1% Nb₂O₅ was measured on disc-shaped specimens (n=5), with a Universal testing machine at a cross-head speed of 0.5 mm/min. The mean biaxial flexural strength was 158.8±37.5 MPa. This value is comparable to previously published values on fluorapatite glass-ceramics commercialized under the name Bioverit®.

The cytotoxicity of the niobium-doped glass-ceramics was evaluated by an Agar diffusion assay adapted from ASTM standard F895-84.137 Human gingival fibroblasts were seeded into six-well culture plates and grown to confluence at 37° C. The culture medium was removed and replaced with a 1:1 mixture of 3% noble agar and 2 MEM. After the agar diffusion layer was formed, neutral red solution was added to the wells and incubated at 37° C. for 30 min. The stain solution was removed and sterile disc specimens (12 mm in diameter, 1.5 mm thick; n=4 per group) were placed on the stained agar surface. A positive control (latex rubber disc) and a negative control (empty well) were included for each plate. The zone of decolorization in which the cells lost their stain was measured and the lysis index calculated. The results revealed that the lysis index of all glass-ceramics was zero on a scale from zero to five, compared to a lysis index of five for the positive control.

### Example 2

### Effect of Heat Treatment Temperature on Microstructure of Fluorapatite Glass-Ceramics

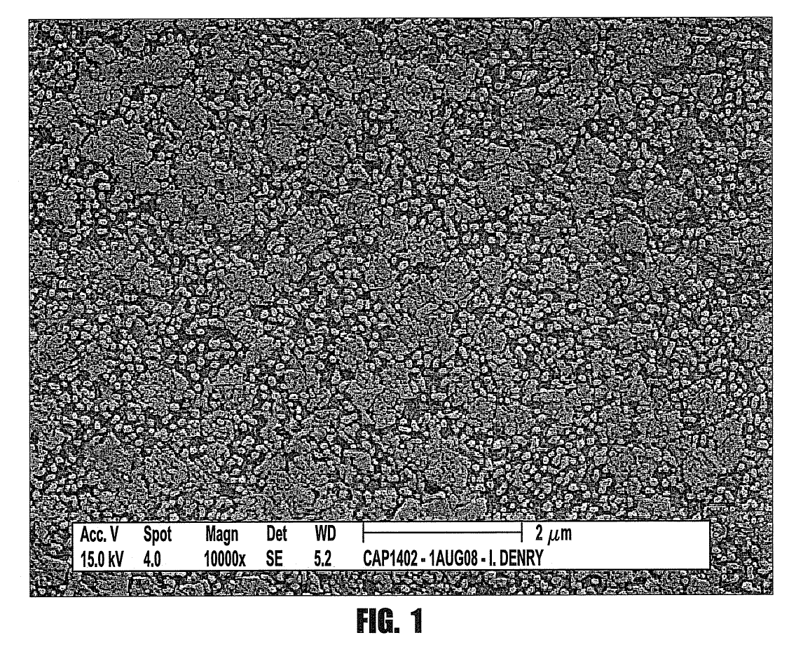

The effect of heat treatment temperature on the microstructure of a fluorapatite-based (FAp) glass-ceramic was investigated in order to optimize the sintering schedule for powder compacts. A fluorapatite-based glass-ceramic composition previously shown to promote crystallization of sub-micrometer crystals was prepared by twice melting at 1475° C. for 3 h. Glass ingots were sectioned into discs (n=3 per group) and heat-treated between 950 and 1200° C. (50° C.-increments) for 1 h. The microstructure was characterized by scanning electron microscopy, quantitative stereology and image analysis. Crystalline phases were analyzed by x-ray diffraction (XRD) on powdered specimens. XRD confirmed the presence of FAp in all specimens, together with forsterite appearing at temperatures above 1000° C. The dual microstructure topography of the FAp glass ceramic is shown in FIG. 1. A dual microstructure of sub-micrometer spherical crystals (SMC) and micron-sized polygonal crystals was observed for heat treatments up to 1050° C. The mean SMC area increased slowly from 950° C. (0.008±0.001 μm²) to 1050° C. (0.022±0.002 μm²) and became significantly larger after heat treatment at 1100° C. or higher (p=0.002), with a mean area of 0.266±0.031 μm² at 1200° C. SMC density per unit area decreased exponentially (R²=0.99) with heat treatment temperature (from 18.7±0.7/μm² at 950° C. to 0.54±0.03/μm² at 1200° C. for 1 h. Polygonal crystals density per unit area also decreased significantly from 0.27±0.01/μm² at 950° C. to 0.12±0.01/μm² at 1200° C. The percent crystallinity did not change significantly after heat treatment up to 1150° C. (65.8±5.1 at 950° C. to 57.0±09 at 1150° C.) but the decrease after heat treatment at 1200° C. (51.7±2.1) was statistically significant (p=0.005). In conclusion, both percent crystallinity and SMC density per unit area decreased with heat treatment temperature, which was attributed to crystal dissolution at high temperature. To encourage formation of a dual microstructure including finely dispersed sub-micrometer fluorapatite crystals, sintering is preferably conducted in the range of 950-1050° C. The surface roughness values can be tailored by modifying the acid etching time, with longer etching times leading to higher surface roughness.

### Example 3

### Characterization of the Sintering Behavior

The sintering behavior of FAp glass powders can be studied using four complementary techniques: dilatometry, real-time ESEM imaging using a heating stage, density measurements and computational modeling of the sintering process. Cylindrical glass pellets (n=3 per group) can be prepared by uniaxial pressing of glass powders. The pellets are subjected to a heat treatment in a horizontal dilatometer (Model 1600D, Orton) at various heating rates (1, 2.5 or 5° C/min.). The initial expansion and sintering shrinkage is recorded as a function of temperature. The sintering behavior can also be assessed by ESEM with an in situ heating stage. Glass particles are placed in a small platinum crucible and heat treated to 975° C. on the heating stage of the microscope. This setup will allow precise determination of the temperature at which neck formation and particle coalescence starts to occur.

The density of the pellets (n=3 per group) is measured before and after sintering using a helium gas displacement pycnometer (AccuPyc II 1340, Micromeritics®). This provides a baseline for the evaluation of the sintering quality of the scaffolds. In addition, a two-scale model can be developed, based on the fact that, in scaffolds, sintering of the strut material can be divided into two distinctive stages. In the early stage, the strut is considered as a powder compact of fine glass particles, while, in a later stage, the strut is considered as a continuous material containing isolated spherical particles. Huang et al., Acta Biomaterialia, 4, 1095-1103 (2008). Separate models are used for the two different states and the transition is assumed to take place when the strut reaches a specific density. These models can assess the effect of simultaneous crystallization on the sintering behavior of the glass-ceramics. The quality of sintering and microstructure can also be assessed by SEM. Qualitative comparisons between model predictions and experimental observations ensure that the sintering schedule is optimized.

### Example 4

### Evaluation of Glass Crystallization

Disc-shaped specimens of glass ceramics are sectioned from the glass ingots, subjected to the nucleation heat treatment, and heat treated in the temperature range 875-975° C. (25° C. increments) for various durations (0.5-2 h). XRD is then be performed on powdered specimens with a first scan at a scanning rate of 1 degree per minute (two-theta) for crystalline phase identification, and a second scan at a scanning rate of 0.2 degree per minute (two-theta) for the determination of the lattice parameters. Alpha alumina can be used as an internal reference standard to ascertain peak position. The crystalline phases can also be analyzed by XRD on bulk specimens, to assess the presence of a surface phase of different crystalline composition. The percent crystallinity is determined by using the Jade x-ray analysis software after background fitting using a spline curve fit. Microstructure can be investigated by both SEM and AFM. Crystallinity, crystal density and crystal dimensions are determined on digital scanning electron micrographs using the public domain NIH Image J program. The results are then compared to those obtained by AFM after image analysis using the AFM software.

### Example 5

### In Vitro Behavior of hMS Cells on Niobium-Doped Fluorapatite Glass-Ceramic

The response of human mesenchymal stem cells (hMSC) to a niobium-doped fluorapatite-based glass-ceramic (FAp) of the present invention was characterized by examining cell spreading behavior, proliferation, and activity. A niobium-doped fluorapatite-based glass-ceramic was prepared by twice melting at 1475° C. for 3 h. The glass was cast into cylindrical ingots that were sectioned into discs (n=3 per group) and heat treated to promote crystallization of fluorapatite submicrometer crystals. hMSCs (Lonza Inc.) were cultured for up to 8 days on FAp glass-ceramic discs and tissue culture polystyrene (TCP) as control. The surface of the FAp discs was either left as-heat treated (HT), ground (GR) or chemically etched (ET). Initial cell attachment was assessed at 3 h. Viability and proliferation data was collected using a live/dead cell viability assay at days 1, 4, and 8. Cell morphology was examined on all surfaces by scanning electron microscopy (SEM) at day 4. Cells were assayed for alkaline phosphatase (ALP) expression at days 1, 4 and 8. There was no significant difference in cell attachment between FAp and control discs (p>0.05). SEM at day 4 revealed the presence of polygonal cells with numerous thin filopodia either attached to the material surface or connected to neighboring cells, regardless of surface state. ALP expression on etched ceramic discs (4.5±0.5 μmol/min/g) was not significantly different than that on the control (3.6±0.6 μmol/min/g). In conclusion, hMSCs displayed excellent attachment, proliferation, and expression on niobium-doped FAp glass-ceramic discs.

### Example 6

### Preparation of Nanocrystalline Fluorapatite Glass-Ceramics Scaffolds

A fluorapatite glass composition containing 5 wt. % Nb₂O₅ was prepared by melting reagent-grade oxides and carbonates at 1525° C. for 3 h as described herein. The molten glass was cast into cylindrical stainless-steel molds and furnace-cooled. The glass was reduced to powder using a planetary ball mill with agate mortar and balls. The grinding cycle was 30 min. at 700 rpm. A ceramic slurry was prepared from this powder dispersed in an aqueous solution containing 1 wt. % polyvinyl alcohol as binder. The optimal solids loading was determined to be 62 wt. %. A 45 ppi (pores per inch) polyurethane foam was impregnated with the slurry following the method described by Schwartzwalder in U.S. Pat. No. 3,090,094. The impregnated foam was then dried at room temperature for 12 h and heat treated at 850° C. for 2 h, with a heating rate of 2° C./min., to burn out the sacrificial polyurethane template. This technique has also been able to fabricate layered scaffolds by impregnating layers of polyurethane foam with different pore sizes, such as a tri-layered scaffold including layers having three different pore sizes.

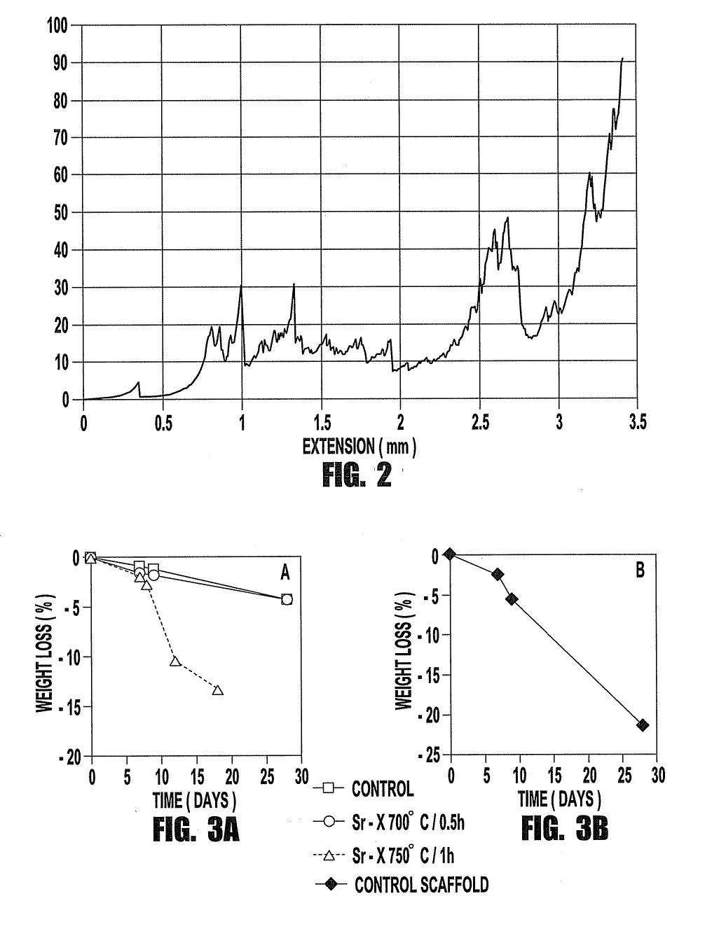

The crystalline phases present were FAp and β-tricalcium phosphate, as determined by XRD. Pilot data on the compressive strength of these scaffolds was obtained using an Instron 4204 testing machine with polished steel compression cylinders at a cross-head speed of 0.5 mm/min. The compressive strength was calculated from the load-deformation curve, by the ratio of ultimate applied force and cross-sectional area of the specimen. Hsu et al., Journal of Materials Science: Materials in Medicine; 18, 2319-2329 (2007). A typical graph showing the strength of the material is shown in FIG. 2. The compressive strength of the scaffold was 1.46 MPa. Scanning electron micrographs of the scaffolds indicated that the pore size was between 150 and 500 μm. The total porosity was 83%, as calculated using the formula:

% P=(1−Wₘ/Wₜₕ)×100

where Wₘ is the measured weight and Wₜₕ the theoretical weight obtained by multiplying the density of the glass by the volume of the sample.

### Example 7

### Preparation of Glass-Ceramics Scaffolds Using Polymer-Coated Foamed Substrate

The polymer replica technique was used to prepare FAp glass-ceramic scaffolds. The replica technique involves pre-coating a polymeric sponge to eliminate sharp apices, slurry impregnation, sintering, and glazing. Crystallization and sintering kinetics will allow the development of a ceramic scaffold with high crystallinity, nanosized fluorapatite crystals and adequate final density and porosity. By pre-coating the template before slurry impregnation, the scaffold strut structure can be improved and laminar defects in the struts can be eliminated by a self-glazing treatment. The combination of these steps will promote a significant increase in compressive strength, leading to a 3D-scaffold with superior structural integrity.

Polyurethane templates are cleaned in distilled water, dried and immersed in NaOH (1 M) for 24 h. Pu et al., J. Am. Ceram. Soc. 90, 2998-3000 (2007) After rinsing in distilled water and drying, the templates are coated with either silica sol (Snowtex, Nissan Chemical) or low viscosity carboxymethyl cellulose (Sigma-Aldrich Inc.). A third experimental group is sputter-coated with carbon (Polaron CC7650 sputter coater), and an additional group is left untreated as a control. The pre-coated templates are characterized by optical microscopy to assess the effect of coating on the morphology of the struts.

The glass-ceramic is reduced to powder with a particle size of less than 45 μin using a planetary ball mill (e.g., a Fritsch® Pulverisette 7) with agate mortar and balls. Slurries are prepared from these powders by dispersion in an aqueous solution containing 1 wt. % polyvinyl alcohol as binder. Three levels of solid loadings from 60 to 80 wt. %, in 10 wt % increments, are tested. The polyurethane templates (45 ppi; 40×12×12 mm) are then impregnated with ceramic slurries. The templates are then be dried for 12 h at room temperature, and further heat treated according to optimal sintering conditions. The sintered scaffolds are then be glazed at 975° C. for 2 min. under vacuum. The vacuum is released as the high temperature is reached. This technique is routinely used to glaze dental porcelains and has been shown to lead to a significant reduction in porosity, from 5.6 to 0.6%. Vines et al., J. Dent. Res., 36, 950-956 (1957).

### Example 8

### Synthesis of Strontium-substituted Apatite by Molten Salt Ion-Exchange

In order to assess the optimal experimental conditions for ion-exchange of strontium for calcium in hydroxyapatite (HAp), tests were conducted using anorganic HAp of bovine origin. Bulk microcrystalline HAp (Clarkson Chromatography Products, Inc., Lot #609121) with a total plate count less than 5,000/G, was mixed with strontium nitrate (99.0%, Alfa Aesar, Ward Hill, Mass.), with a salt to HAp ratio of 1.6 using the method of Tas. Tas, A. C., Journal of the American Ceramic Society, 84, 295-300 (2001). Several HAp/salt ratios were investigated, but only is reported here for brevity. The mix was heat treated at 900° C. for 30 minutes in a covered alumina crucible and furnace-cooled to room temperature. The salt was then eliminated by repeated rinsing in distilled water until no remaining salt was detectable by XRD, which also revealed the formation of fully substituted strontium-apatite.

The successful exchange of calcium for strontium in anorganic HAp helped set the basis for ion-exchange of Nb-doped FAp glass-ceramics. Glass disks were heat treated at 950° C./2 h, to simulate scaffold sintering. Specimens were then placed in alumina crucibles, covered with strontium nitrate, heat treated at temperatures ranging between 650 and 900° C./30 min., and furnace-cooled to room temperature. Strontium nitrate was eliminated by repeated rinsing in distilled water. The bulk surface of the specimens was analyzed by XRD. Partial substitution of strontium for calcium was observed after ion-exchange above 700° C. for 30 min. and 750° C. for 1 h, while no exchange was observed after heat treatment at 650° C. for 1 h. The extent of the substitution was estimated by linear regression using the position of the three most intense reflections and published data on strontium-substituted apatites. Bigi et al., Materials Science Forum; p 814-819 (1998).

The inventors established that the mild ion-exchange conditions initially used (700° C./0.5 h) only led to a 5 micrometer-thick exchanged layer of partially substituted (75%) Sr-apatite, while after ion-exchange at 750° C./1 h, XRD revealed the presence of partially substituted (81%) Sr-apatite and a depth of exchange of at least 24 micrometers. The depth of exchange was estimated by sequential grinding of the surface until XRD diffraction detected only fluorapatite. These experiments confirm that the depth and quality of the exchange is proportional to the temperature and duration of heat treatment.

### Example 9

### Solubility of Fluorapatite and Strontium-Substituted Apatite Glass-Ceramics

The solubility of FAp glass-ceramics doped with 1 wt. % niobium oxide was evaluated according to ISO 6872143, after aging in acetic acid at 80° C. The weight loss of disk-shaped specimens of the untreated control and fluorapatite glass-ceramic after strontium-exchange at 700° C./0.5 h and 750° C./1 h, as well as an untreated scaffold specimen was measured over a period of 18 days to 4 weeks. The results (FIGS. 3A and B) show that the control scaffold specimen had the greatest solubility, with a 22% weight loss at 30 days (3B). The weight loss at 7 and 9 days was 50% and 66% greater, respectively, for the bulk specimen exchanged at 700° C. for 0.5 h than for the untreated control specimen. The weight loss at 30 days was similar. However, as shown in FIG. 3A, the weight loss at 18 days was 4.5 greater after ion-exchange at 750° C./1 h than after ion-exchange at 700° C./0.5 h, indicating that the depth and quality of the exchange is proportional to the temperature and duration of heat treatment. Based on these results, it is clear that the solubility of the scaffold can be varied by simply adjusting ion-exchange depth and degree of substitution through temperature and duration of heat treatment. A thin exchanged-layer increased solubility only at the beginning of the experiment, and later led to a tapering of the weight loss, as this layer dissolved. The inventors expect that a thicker ion-exchanged layer will further increase weight loss and that the solubility of FAp glass-ceramics can be adjusted by ion-exchange with strontium.

### Example 10

### Characterization of Strontium-Exchanged Fluorapatite Glass-Ceramics by X-Ray Diffraction

The crystalline phases and degree of exchange in fluorapatite glass-ceramics after ion-exchange was characterized at various temperatures. A fluorapatite-based glass-ceramic was prepared by twice melting at 1475° C. for 3 h. The glass was cast into cylindrical ingots that were sectioned into discs (n=3 per group) and heat treated to promote crystallization of fluorapatite. The discs were further treated by ion-exchange in molten strontium salt at temperatures between 600 and 700° C. (in 25° C.-increments) for 1 h. Treated discs were cleaned and analyzed by x-ray diffraction (XRD) on bulk surfaces. XRD was also performed after sequential grinding to assess the depth of exchange as a function of temperature. The phase composition was determined using Jade XRD software, together with available diffraction data on Sr-fluorapatite. XRD analyses revealed the formation of partially exchanged strontium fluorapatite. The presence of reflections corresponding to fluorapatite for heat treatments at temperatures below 700° C. and after sequential grinding was revealed by deconvolution. The degree of strontium for calcium exchange was between 35 and 40% and appeared independent of the ion-exchange temperature. The corresponding lattice parameters were within the range of published diffraction data for Sr-fluorapatite. The intensity ratio of the (112) to the (211) reflections (corresponding to the crystallographic Miller indices) for partially exchanged Sr-fluorapatite increased linearly with increasing the ion-exchange temperature (from 0.55±0.03 at 600° C. to 0.73±0.01 at 700° C.; R²=0.97). This increase was statistically significant (p=0.001). The depth of the ion-exchanged layer increased with treatment temperature and was estimated at 10 micrometers after heat treatment at 600° C. for 1 h, and up to 80 micrometers after heat treatment at 700° C. for 1 h. In conclusion, heat treatment of fluorapatite glass-ceramic in molten strontium salt led to partial ion-exchange of strontium for calcium. The depth of exchange increased with heat treatment temperature.

### Example 11

### Evaluation of the Resorption and Bone Regeneration Ability of Ceramic Scaffolds In Vivo Using a Rat Calvarial Defect Model

The well-established rat calvarial defect model has long been used to evaluate and quantify bone regeneration. Dahlin et al., J. Neurosurg. 74, 487-491 (1991) The amount of newly formed bone depends on the size of the defect. The critical size has been defined as the defect size for which there is no spontaneous healing during the life time of the animal. Previous studies in the rat model have shown that a defect size of 8.8 mm in diameter in rats meets this criteria. Honma et al., Oral Diseases, 14, 457-464 (2008).

The following scaffold materials will be tested: a) Glass-ceramic scaffolds of the present invention b) commercial bone graft substitute (Vitoss®, Orthovita Inc.) c) particulate autogenous bone: positive control d) no scaffold: negative control. The choice of a β-TCP (Vitoss®) commercial bone graft substitute is justified by the fact that there is no currently available HAp or bioactive glass scaffold material that can be shaped and offers pore size and percent porosity comparable to the glass-ceramic scaffold material of the present invention. Previous studies using the same defect size in rats have shown that a standard deviation (σ) of about 10% can be expected for new bone formation at 12 weeks and a minimum difference (Δ) of 20% is defined as our goal to detect. A sample size (n) of 9 animals per group was determined to be adequate.

A total of 126 male (inbred) Sprague-Dawley rats (weight 200-300 g) are used for the study. Surgery is performed under general inhalation anesthesia. An incision is made along the sagittal plane of the cranium and a full-thickness flap is reflected, exposing the calvarial bone. A standardized full thickness bone defect, 8.8 mm in diameter is trephined in the center of the parietal bone under constant saline irrigation and without damaging the dura. The removed calvarial disks are then milled in a bone mill and used as autogenous bone graft positive control. The animals are randomly assigned to one of the five treatment groups and two control groups. The periosteum and skin is then be closed and sutured.

Intravital calcein and alizarin bone labels (Sigma, St Louis, Mo.; 30 mg/kg and 20 mg/kg, respectively) are administered i.p. in saline vehicle 10 and 7 days prior to sacrifice to mark new forming bone surfaces. New bone formation are measured by in vivo micro-computed tomography under general anesthesia (micro-CT; Inveon™ Siemens-medical) at 0, 1, 4, 8, 12, 24, 36 weeks. Several studies have evaluated the radiation exposure during micro-CT imaging in rodents. Figueroa et al., Medical Physics 35, 3866-3874 (2008) It was shown that micro-CT imaging radiation doses do not produce gross tissue histology changes. Ford et al., Medical Physics, 30, 2869-2877 (2003)

Micro-CT computer software allows the determination of bone quality such as connectivity density and SMI, as well as 3-D image reconstruction. A baseline scan at the time of implantation and administration of bone labels allows careful monitoring of new bone formation as well as scaffold resorption. Half of the animals are sacrificed at 12 weeks, the other half at 36 weeks. Following euthanasia, the grafts and surrounding cranial tissue are retrieved en bloc and prepared for histological evaluation. The specimens are fixed in 10% cold neutral buffered fonnalin, dehydrated in a graded series of ethanol solutions and embedded in methyl methacrylate. After polymerization, 5-micrometer thick transverse sections are made by using a modified microtome technique. Van der Lubbe et al., Stain Technol. These sections are then stained with McNeal's stain TRAP and methylene blue for cell characterization by light microscopy. Qualitative analysis of the sections will include assessment of eventual inflammatory reactions and/or fibrous capsule and identification of new bone. In addition, thick sections (80 microns) will allow for measurement of intravital bone labels. Traditional histomorphometric variables (BV/TV, MAR, MS/BS and BFR) can be measured using Bioquant software. Image analysis is performed on three histological sections per graft site. Calcein and alizarin bone labels allow one to determine the location of anabolic activity after examination under epifluorescence microscopy.

The complete disclosure of all patents, patent applications, and publications, and electronically available materials cited herein are incorporated by reference. Any disagreement between material incorporated by reference and the specification is resolved in favor of the specification. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A bioactive and bioresorbable scaffold, comprising:
a glass-ceramic material shaped into a scaffold comprising fluoroapatite and hydroxyapatite doped with about 1-5 wt. % niobium oxide, wherein the glass-ceramic material has high crystallinity and a complex topography.

2. The scaffold of claim 1, wherein the glass-ceramic material has a flexural strength of at least about 100 MPa, a modulus of elasticity of at least about 80 GPa, and a fracture toughness of at least about 1.2 MPa.m^{0.5}.

3. The scaffold of claim 1, wherein the glass-ceramic material includes an outer layer comprising strontium.

4. The scaffold of claim 3, wherein the glass-ceramic material includes an outer layer has a thickness from about 5 micrometers to about 25 micrometers.

5. The scaffold of claim 1, wherein the scaffold is configured for restoring or regenerating bone, cartilage, muscle, or musculoskeletal tissue.

6. The scaffold of claim 1, wherein the glass-ceramic has a crystallinity of at least about 55%.

7. The scaffold of claim 1, wherein the complex topography comprises nanosized fluoroapatite crystals having a surface density of from about 2 to about 25 per square micrometer.

8. The scaffold of claim 7, wherein the crystals have a size ranging from about 50 to about 400 nanometers.

9. The scaffold of claim 8, wherein the complex topography comprises fluoroapatite crystals having a size from about 80 to 100 nanometers and larger crystals comprising forsterite having a size ranging from about 300 to about 1000 nanometers.

10. The scaffold of claim 1, wherein the complex topography stimulates osteogenesis.

11. A method of musculoskeletal engineering comprising positioning a bioactive and bioresorbable scaffold according to claim 1 in a subject to provide structural support for nearby tissue.

12. The method of claim 11, wherein the method is used in oral or maxillo-facial surgery.

13. A method of making a bioactive and bioresorbable scaffold, comprising the steps of:
Melting suitable reagent grade oxides and carbonates together with niobium oxide at a temperature from about 1450 to 1600° C. to obtain a glass-ceramic material comprising: 28-38% SiO₂, 12-18% CaO, 12-18% MgO, 11-17% Al₂O₃, 1-3% Na₂O, 5-8% K₂O. 4-6% F, 10-14% P₂O₅, and 1-5% Nb₂O₅, and then allowing the glass-ceramic material to cool;
Grinding the glass-ceramic material to a powder and then remelting the glass-ceramic material at a temperature from about 1450 to 1600 C to homogenize the glass ceramic material, and again allowing it to cool;
Grinding the glass-ceramic material to a powder and compacting and sintering the glass-ceramic material at a temperature from about 750 to about 1100° C. and allowing it to cool to form a glass-ceramic scaffold.

14. The method of claim 13, wherein the glass-ceramic material is sintered over a polymeric foam suitable for forming a porous glass-ceramic scaffold.

15. The method of claim 14, wherein the polymeric foam comprises a pre-coat comprising silica sol or carboxymethyl cellulose.

16. The method of claim 13, wherein the glass-ceramic scaffold is provided with an outer layer comprising strontium by ion-exchange.

17. The method of claim 16, wherein the ion-exchange is carried out using molten strontium nitrate at a temperature from about 650 to about 800° C.

18. The method of claim 16, wherein the ion-exchange is carried out using a mixture of molten strontium nitrate and strontium dinitrate at a temperature from about 550 to about 650° C.

19. The method of claim 16, wherein the solubility of the glass-ceramic scaffold can be increased by increasing the depth of the outer layer comprising strontium.

20. A bioactive and bioresorbable scaffold prepared according to the method of claim 13.
